(19) United States
(12) Patent Application Publication
(10) Pub. No.: US 2005/0187479 A1
(43) Pub. Date: Aug. 25, 2005

(54) Cable-free endoscopy method and system for determining in vivo position and orientation of an endoscopy capsule

(76) Inventor: Rainer Graumann, Hochstadt (DE)

Correspondence Address: SCHIFF HARDIN, LLP;PATENT DEPARTMENT, 6600 SEARS TOWER, CHICAGO, IL, 60606-6473, US

(21) Appl. No.: 11/015,357

(22) Filed: Dec. 17, 2004

(30) Foreign Application Priority Data
Dec. 19, 2003 (DE) 103 59 981.9

(51) Int. Cl. A61B 6/00

(52) U.S. Cl. 600/476

(57) ABSTRACT

In a system and method for determination of in-vivo orientation positions of an endoscopy capsule in the context of cable-free endoscopy, a magnetic field generator generates an electromagnetic HF-3D gradient field in the examining region, an endoscopy capsule has an integrated detector that ascertains the current respective position-specific and orientation-specific HF-3D gradient field values of the endoscopy capsule, a navigation unit is supplied with those values and assigns spatial information to the current HF-3D gradient field values, and a visualization unit displays the anatomical images acquired in a defined endoscopy capsule position and orientation on a monitor.

### BACKGROUND OF THE INVENTION

The present invention relates generally to cable-free endoscopy using an endoscopy capsule, such as an endo-robot, for execution of minimally invasive diagnosis and intervention in the inner body, such as in the gastrointestinal tract of a patient. The present invention particularly concerns a system as well as a method for determination of in-vivo positions of the endoscopy capsule.

### DESCRIPTION OF THE PRIOR ART AND RELATED SUBJECT MATTER

A cable-free endoscopy capsule 1 has recently come into use in endoscopic diagnosis, as depicted in FIG. 1. (The endoscopic capsule 1 shown in FIG. 1 is an embodiment of the invention, but can be used to explain the components thereof that are known.) The capsule 1 has a camera 3, an internal voltage supply, a memory module 19 as well as a transmitting device 4 with an antenna 5. The endoscopy capsule 1 is taken orally by the patient and then traverses the intestinal tract in a natural way. The endoscopy capsule 1 is able to transmit images (for instance, one image per second), obtained continuously by the camera 3 from the intestinal tract to the outside by means of the internal transmitting device 4 and the antenna 5. The images are received by an antenna 10 of a receiver unit 9 and saved and finally displayed on a visualization unit 8 according to FIG. 2.

A cable-free endo-robot is also known from U.S. Pat. No. 6,240,312 that, in comparison to the endoscopy capsule 1, additionally features an ablation-capable laser 7 and moreover can be actively steered from outside by controlled variation of external magnetic fields. The steering of such an endo-robot 2 in the inner body is achieved with a magnetic field steering system and is extensively described in German Patent 101 42 253.

The endo-robot 2 is provided with a linear magnet (bar magnet or linear coil 6) that reacts to variable 3D gradient magnetic fields applied from the exterior causing a linear force and torque to act on the endo-robot for actively steering the endo-robot 2, namely for remote-controlled movement and orientation in the inner body. The user navigates the endo-robot 2 by pitch from front to back and right to left as by forward, backward and sideways motion, as well as of an input lever 17, via a pressure input device 16, for instance a so-called 6D mouse. The images taken by the endo-robot camera 3 are transmitted to the exterior of the body by means of the transmitting device 4 and the antenna 5 and are displayed on the visualization unit 8 after reception by the antenna 10 and the receiver unit 9.

As noted, the endoscopy capsule 1 and endo-robot 2 are equipped with an integrated (mini) camera, that continuously transmits an image from the inner body on a controlled path through the intestinal tract, possibly for several hours (6 to 8 hours). Therewith, endoscopic viewing of the entire small intestine is made possible with a very high discovery rate for pathological changes.

A problem, however, is to make a correct correlation to anatomical structures, of an image obtained by the endoscopy capsule 1 or endo-robot 2 in a given position and orientation. An image mapping conventionally takes place based solely upon the available anatomical knowledge of the user.

### SUMMARY OF THE INVENTION

An object of the present invention is to provide a system and a method to directly assign the position and orientation of an endoscopy capsule or endo-robot to a point in time with the acquisition of the image by the endoscopy capsule or endo-robot.

This object is achieved in accordance with the invention by a system for determination of in-vivo positioning and orientation of an endoscopy capsule cable-free endoscopy, having a magnetic field generator that generates electromagnetic HF-3D gradient fields in the region to be examined, an endoscopy capsule with a detector unit for integrated therein ascertain respective actual position-specific and orientation-specific HF-3D gradient field values of the endoscopy capsule, a navigational unit that is supplied with these values and assigns spatial information to the current HF-3D gradient field values, and a visualization unit that displays on a monitor anatomical images acquired at a defined endoscopy capsule position and orientation.

The endoscopy capsule can be designed as an endo-robot.

In an embodiment of the invention the endoscopy capsule contains a memory module that stores the corresponding position-specific and orientation-specific HF-3D gradient field values for a particular anatomical image acquisition, that are then read by the navigation unit.

In another embodiment of the invention the endoscopy capsule contains a transmitting unit that transmits the position-specific and orientation-specific HF-3D gradient field values for anatomical image directly to the navigation unit.

The integrated detector unit preferably is a sensor coil.

In a further embodiment the magnetic field generator is portable and is attached to the patient, who carries it with him or her over a longer period of time.

Instead of transmitting directly to the navigation unit, the transmitter in the endoscopy capsule can transmit the values to a receiver unit that in turn supplies the values to the navigation unit.

In this case it is also useful to make the receiver unit portable as well and to attach the receiver unit to the patient as well.

According to the present invention the navigation system makes an assignment of the anatomical images to the respective position and orientation of the endoscopy capsule possible.

According to the present invention the display of the endoscopy capsule takes place on the visualization unit.

The visualization unit enables a parallel display of the endoscopy capsule and the anatomical image obtained in this position and orientation in a further embodiment of the invention.

A method in accordance to the invention ascertaining the in-vivo position and orientation of an endoscopy capsule in cable-free endoscopy is includes the steps of generating an electromagnetic HF-3D gradient field in the examining region by means of a magnetic field generator, acquiring respective current position-specific and orientation-specific HF-3D gradient field values of the endoscopy capsule with a detector unit, assigning spatial information to the current HF-3D gradient field values by means of a navigation unit, and visually displaying the anatomical images obtained in a defined position and orientation on a monitor.

The method can include the steps of integrating the detector unit into the endoscopy capsule.

The method also can include the steps of designing the endoscopy capsule as an endo-robot.

The method also can include the step of storing the position-specific and orientation-specific HF-3D gradient field values of the endoscopy capsule corresponding to the respective anatomical image acquisition in a memory component.

The method also can include the step of transmitting the position-specific and orientation-specific HF-3D gradient field values corresponding to the respective anatomical image acquisition to the navigation unit by means of a transmitter unit integrated into the endoscopy capsule.

A sensor coil can be used as an integrated detector unit in the inventive method.

The method can include the step of transmitting the values from the endoscopy capsule to a receiver unit, and from these to the navigation unit.

The endoscopy capsule can be displayed on the visualization unit as well in the inventive method.

The method also can include the step of attaching the magnetic field generator and the receiver unit (if used) as well to the patient by whom it will be carried in a portable manner over a longer period of time.

The method can include the step of assigning of anatomical images to respective endoscopy capsule position and orientation by the navigation unit.

### DESCRIPTION OF THE DRAWINGS

FIG. 1 schematically illustrates an endoscopy capsule in accordance with the invention with an integrated camera, a memory module as well as a transceiver unit and an antenna for image broadcasting.

FIG. 2 depicts a visualization unit with antenna and detector unit for display of images.

FIG. 3 depicts an endo-robot in accordance with the invention with an integrated camera, a memory module, a transceiver unit and an antenna for image broadcasting as well as with an ablation capable laser and linear magnet for active navigation.

FIG. 4 depicts an endoscopy capsule in accordance with the invention with a magnetic field generator, receiver unit, and a navigation unit in the small intestine of a patient.

FIG. 5 depicts an endo-robot in accordance with the invention with magnetic field control system and navigation unit in the small intestine of a patient.

### DESCRIPTION OF THE PREFERRED EMBODIMENTS

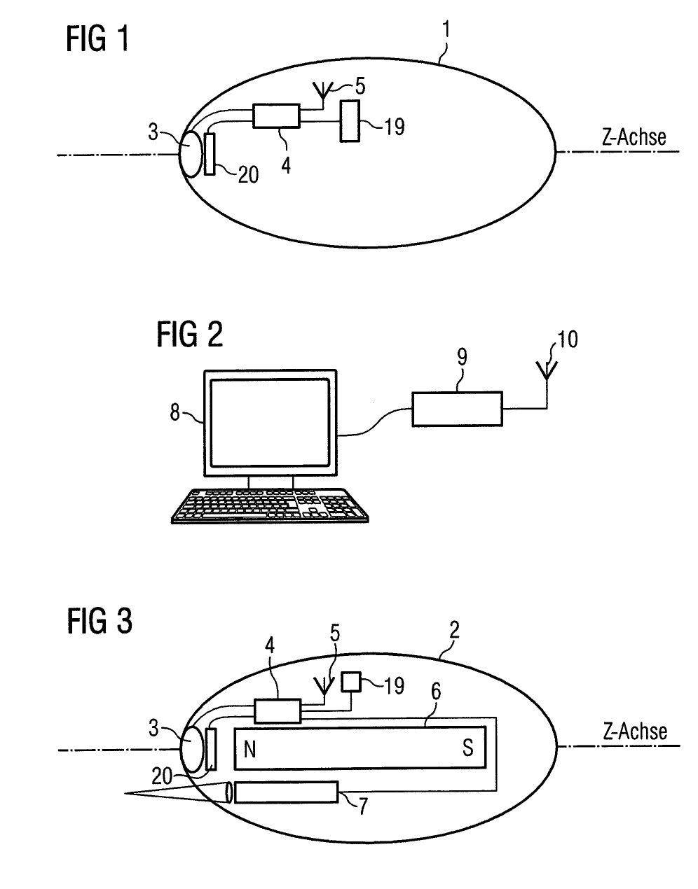

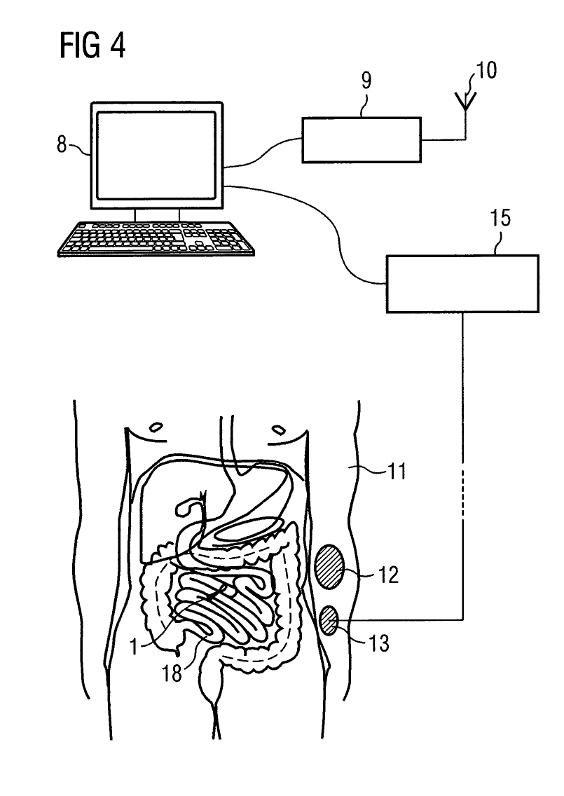

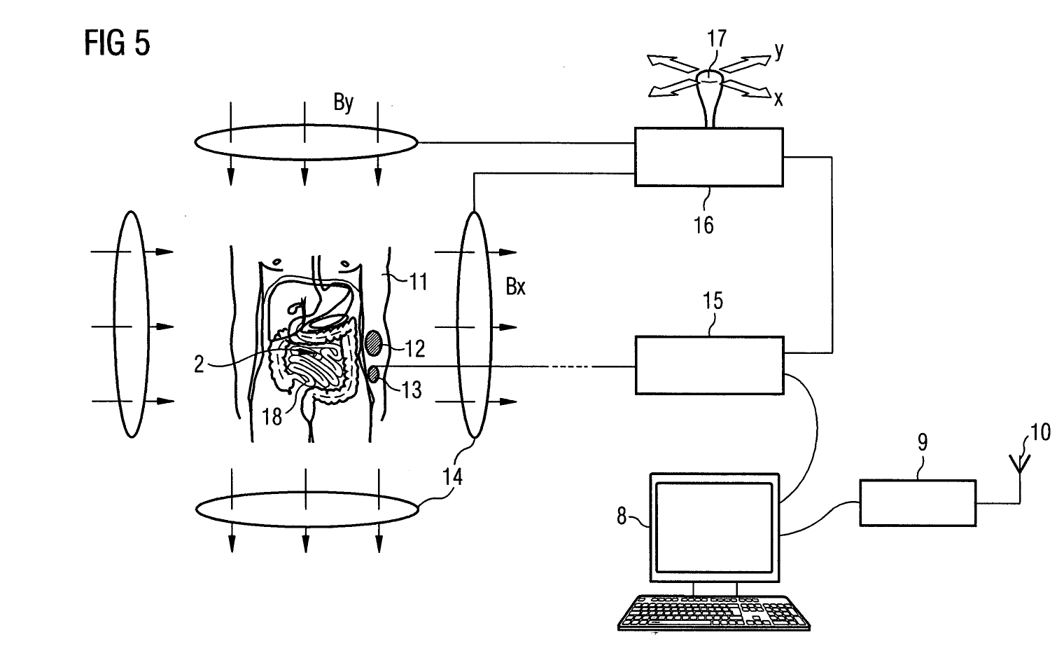

The present invention concerns a system, and a method that enable the assignment of an acquired image of camera 3 to the respective spatial position and orientation of the endoscopy capsule or an endo-robot existing at the time the image was acquired, during image acquisition. An endoscopy capsule is shown in FIGS. 1-4, and the endoscopy capsule is designed as a navigational endo-robot in the embodiment of FIG. 5.

The system according to the present invention includes a magnetic field generator 12, which is located outside of the body and generates a strong, variable magnetic alternating field in the region in which the endoscopy capsule or the endo-robot moves and acquires images (for example, a gradient field or a quadrupole field).

The frequency of this alternating magnetic field is on the order of several kHz, so that this alternating magnetic field penetrates the human body nearly undisturbed. The endoscopy capsule or the endo-robot is equipped with a sensor coil 20 which is dimensioned (so that the sensor coil 20) detects the alternating magnetic field. The sensor coil 20 exhibits five or six gyros that emit signals dependent on the strong, varying magnetic alternating fields, as it is spatially varied (adjusted). The measurement defines the position and orientation of the sensor coil 20 in the magnetic alternating field, and thus the position and orientation of the endoscopy capsule or endo-robot, relative to the body or relative to the surrounding space.

In this way an unambiguous relationship between the endoscopy capsule the endo-robot, and the anatomy can be established. This is meaningful and important to allow diagnosed injuries or pathologies of the intestine to be located and from the exterior of the body and treated with minimally invasive therapies.

The unambiguous relationship between the position and orientation of the sensor coil 20 and the anatomy is established via a conventional registration method. Anatomical landmarks within the patient (bones, organs) that in which the measuring of supporting anatomical images of differing image producing means (C-arch, ultrasonic, magnetic resonance tomography, etc.) can be identified as well are chosen by the user. An external navigation unit 15 makes a calculational linking of the signal of sensor 20 to the registration defined coordinate system. In this way it is theoretically possible to determine with an accuracy of approximately 1 mm the position and orientation of the sensor coil 20 and thus the endoscopy capsule 1 or the endo-robot 2, via the registration defined coordinate system. However, only an orienting accuracy of several centimeters is practical. Due to the ever-present organ movement a time-dependent discrepancy between the anatomy at the point in time of the registration and at the point in time of measuring by the sensor coil 20 exists.

The signal of the sensor coil 20, which defines the exact position and orientation in the alternating magnetic field, can be transmitted immediately to an external receiver unit 13, and from this conducted further to the navigation unit 15 and fed to the visualization unit 8, so that for every acquired image of the endoscopy capsule or endo-robot, the respective orientation and position of the endoscopy capsule or the endo-robot can be depicted on the same monitor screen. Storage of the signal from the sensor coil 20 in an internal memory module 19 of the endoscopy capsule or the endo-robot 2 is also possible, which then are transmitted directly to the navigation unit 15. In this case the receiver unit 13 can be dispensed with.

In a further embodiment of the invention can carry the magnetic field generator as well as the receiver unit (if used) on a belt, allowing signals to be obtained over several hours. This is meaningful, if a time period in a rest position cannot be expected of the patient, for example, for the exclusive application of endoscopy capsule. The duration is dependent on the paristalsis of the digestive organs, and spans multiple hours, alternately days.

It should be noted, that the frequencies of the 3D gradient fields used in the magnetic field steering control system are in the low Hz range and in no way influence the high frequency magnetic alternating field of the the position recognition system, let alone cause damage. However, in case troublesome interference occur, it would be possible to filter the low frequencies out of the high frequency alternating magnetic field.

Although modifications and changes may be suggested by those skilled in the art, it is the intention of the inventor to embody within the patent warranted hereon all changes and modifications as reasonably and properly come within the scope of his contribution to the art.

1. A cable-free endoscopy system comprising:
an endoscopy capsule adapted for in-vivo manipulation in a body of a patient;
a magnetic field generator that generates a high-frequency, three-dimensional electromagnetic gradient field in a region of the patient in which said endoscopy capsule is disposed;
a detector integrated in said endoscopy capsule that interacts with said gradient field and generates respective position-specific and orientation-specific values of said gradient field for said endoscopy capsule;
a navigation unit supplied with said values, said navigation unit assigning spatial information to said values; and
a visualization unit including a monitor for displaying anatomical images acquired by said endoscopy capsule with a correct position and orientation dependent on said position-specific and orientation-specific values.

2. A system as claimed in claim 1 wherein said endoscopy capsule is an endo-robot.

3. A system as claimed in claim 1 comprising a memory contained in said endoscopy capsule in which said position-specific and orientation-specific values are stored, in a manner readable by said navigation unit.

4. A system as claimed in claim 1 comprising a transmitter unit in said endoscopy capsule for transmitting said position-specific and orientation-specific values to an exterior of the patient.

5. A system as claimed in claim 4 comprising a receiver unit that receives said values transmitted by said transmitter unit, said receiver unit forwarding said values to said navigation unit.

6. A system as claimed in claim 5 wherein said receiver unit is a portable receiver unit and wherein said magnetic field generator is a portable magnetic field generator, each of said portable receiver unit and said portable magnetic field generator being adapted to be worn by the patient.

7. A system as claimed in claim 1 wherein said magnetic field generator is a portable magnetic field generator adapted to be worn by the patient.

8. A system as claimed in claim 1 wherein said detector unit comprises a sensor coil.

9. A system as claimed in claim 1 wherein said visualization unit displays a representation of said endoscopy capsule.

10. A system as claimed in claim 9 wherein said visualization unit displays said representation of said endoscopy capsule in parallel with said anatomical images.

11. A cable-free endoscopy method comprising the steps of:
introducing an endoscopy capsule adapted for in vivo manipulation into a body of a patient;
generating a high-frequency, three-dimensional electromagnetic gradient field in a region of the patient in which said endoscopy capsule is disposed;
introducing a detector in said endoscopy capsule that interacts with said gradient field and generates respective position-specific and orientation-specific values of said gradient field for said endoscopy capsule;
supplying said values to a navigation unit external to the patient, and in said navigation unit assigning spatial information to said values; and
displaying anatomical images acquired by said endoscopy capsule on a monitor with a correct position and orientation dependent on said position-specific and orientation-specific values.

12. A method as claimed in claim 11 comprising employing an endo-robot as said endoscopy capsule.

13. A method as claimed in claim 11 comprising storing said position-specific and orientation-specific values in a memory in said endoscopy capsule, in a manner readable by said navigation unit.

14. A method as claimed in claim 11 comprising transmitting said position-specific and orientation-specific values from said endoscopy capsule to an exterior of the patient from a transmitter in said endoscopy capsule.

15. A method as claimed in claim 14 comprising receiving said values transmitted by said transmitter, and forwarding said values to said navigation unit.

16. A method as claimed in claim 15 comprising receiving said values with a portable receiver unit and generating said magnetic field generator with a portable magnetic field generator and attaching each of said portable receiver unit and said portable magnetic field generator being so as to be worn by the patient.

17. A method as claimed in claim 11 comprising generating said magnetic field generator is a portable magnetic field generator worn by the patient.

18. A method as claimed in claim 11 comprising employing a sensor coil as said detector unit.

19. A method as claimed in claim 11 comprising displaying a representation of said endoscopy capsule on said monitor.

20. A method as claimed in claim 19 comprising displaying said representation of said endoscopy capsule in parallel with said anatomical images on said monitor.
